(19) United States
(12) Patent Application Publication
(10) Pub. No.: US 2018/0161219 A1
(43) Pub. Date: Jun. 14, 2018

(54) DECORATIVE DISPOSABLE UNDERGARMENT

(71) Applicant: Yvonne I. Howe, Marshall, MI (US)

(72) Inventor: Yvonne I. Howe, Marshall, MI (US)

(21) Appl. No.: 15/373,678

(22) Filed: Dec. 9, 2016

(51) Int. Cl. A61F 13/74

(52) U.S. Cl. CPC A61F 13/74; A61F 2013/8497

(57) ABSTRACT

A decorative disposable undergarment which having features which make it a stronger, more fitted, and more aesthetic. The decorative disposable undergarment has a undergarment body formed from a disposable brief style undergarment that is constructed of a cotton-like fabric and includes a sewn in pad and a plurality of elastic, decorative retaining strips which each encircle the undergarment body and are attached to the exterior surface thereof. The strips, which each include a decorative design, are positioned on the undergarment body so that collectively, the strips extend from the top edge of the undergarment body to just above the uppermost portion of the pad. Attached to and extending down from the third strip is a hanging portion which operates to extend the decorative indicia further down the surface of the undergarment body.

### BACKGROUND OF THE INVENTION

### Field of the Invention

This invention relates generally to nether garments and, more particularly, to a disposable incontinence undergarment having decorative retaining strips.

### Description of the Prior Art

The use of contemporary disposable incontinence undergarments for absorbing urine or feces of a wearer while being worn is well known. Unlike traditional disposable incontinence undergarments, which were often bulky and plastic based, many contemporary disposable incontinence undergarments such the Depend® FIT-FLEX® and Silhouette® lines, are constructed of fabric like material and give the appearance of a normal fabric underwear. A problem which still exists, however, is that while such contemporary options many improve the appearance of disposable incontinence undergarments when viewed from outside ones clothing, they still generally are unable to mimic the aesthetics of conventional fabric undergarments. For example, while it is often desirable for women to wear lace garments in order to enhance ones desirability to a partner, it is generally believed to be unfeasible to have a lace design for disposable incontinence undergarments. Thus, there remains a need for a decorative disposable undergarment which includes a plurality of retaining strips which display decorative indicia. It would be helpful for such a decorative disposable undergarment to additionally employ the decorative retaining strips to compress the undergarment into a wearer's body. It would be additionally desirable for such a decorative disposable undergarment to additionally employ the decorative retaining strips for reinforcing the undergarment body.

The Applicant's invention described herein provides for a decorative disposable undergarment adapted to allow a user to comfortably wear a decorative in appearance undergarment that is disposable. The primary components in Applicant's decorative disposable undergarment are an undergarment body and a plurality of decorative retaining strips. When in operation, the decorative disposable undergarment enables an incontinent user to feel better about themselves by wearing a garment that is visually attractive to themselves and a partner. As a result, many of the limitations imposed by prior art structures are removed.

### SUMMARY OF THE INVENTION

A decorative disposable undergarment which having features which make it a stronger, more fitted, and more aesthetic. The decorative disposable undergarment comprises a undergarment body defining a disposable brief style undergarment that is constructed of a cotton-like fabric and includes a sewn in pad and a plurality of elastic, decorative retaining strips which each encircle the undergarment body and are attached to the exterior surface thereof. The strips, which each include a decorative design, are positioned on the undergarment body so that collectively, the strips extend from the top edge of the undergarment body to just above the uppermost portion of the pad. Attached to and extending down from the third strip is a hanging portion which operates to extend the decorative indicia further down the surface of the undergarment body.

It is an object of this invention to provide a decorative disposable undergarment which includes a plurality of retaining strips which display decorative indicia.

It is another object of this invention to provide a decorative disposable undergarment which additionally employs the decorative retaining strips to compress the undergarment into a wearer's body.

It is yet another object of this invention to provide a decorative disposable undergarment which additionally employs the decorative retaining strips for reinforcing the undergarment body.

These and other objects will be apparent to one of skill in the art.

### BRIEF DESCRIPTION OF THE DRAWINGS

FIG. 1 is a side perspective view of a decorative disposable undergarment built in accordance the present invention.

### DETAILED DESCRIPTION OF THE INVENTION

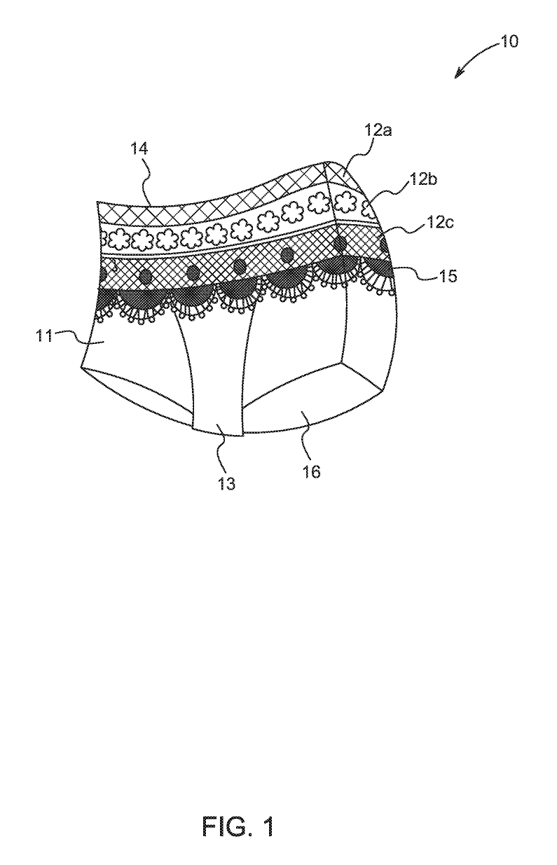

Referring now to the drawings and in particular FIG. 1, a decorative disposable undergarment 10 for a woman is shown having a undergarment body 11 and a plurality of decorative retaining strips 12a, 12b, 12c (collectively, 12). As with contemporary disposable incontinence undergarments, the undergarment body is constructed of a cotton-like fabric. In one embodiment, the undergarment body 11 defines a disposable brief style undergarment that includes a sewn in pad 13.

In order to improve the durability of the undergarment body 11 as well as its ability to remain close to a wearer's body, a plurality of decorative retaining strips 12 are positioned thereon so as to encircle the undergarment body 11. In one embodiment, the strips 12 are constructed of a knitted elastic. It is appreciated that as knitted elastics can be customized with stripes, patterns, and prints, such a decoration on the knitted elastic will define the decorative indicia displayed by each strip 12.

The strips 12 are positioned on the undergarment body 11 so that collectively, the strips 12 extend from the top edge 14 of the undergarment body 11 to just above the uppermost portion of the pad 13. In this regard, FIG. 1 shows a first strip 12a that extends down from the top edge 14 of the undergarment body 11, replacing a typical elastic waistband, a second strip 12b extending downward from just under the lowermost edge of the first strip 12a, and a third strip 12c extending downward from just under the lowermost edge of the second strip 12b.

Attached to and extending down from the third strip 12c is a decorative hanging portion 15 which operates to extend the decorative indicia further down the surface of the undergarment body 11. In one embodiment, the hanging portion 15 may be constructed of the same type of material as a lingerie, such as a satin or lace, to give the decorative disposable undergarment 10 a more authentic appearance.

In one embodiment, the strips 11 are attached to the exterior surface of the undergarment body 11. In this regard, it is contemplated that the strips 11 may be sewn to the undergarment body 11.

In the illustrated embodiment, each strip 12 is shown having a different design. It is appreciated that in other embodiments, the strips 12 may alternatively have identical, similar, or complementary designs.

It is appreciated that while the strips 12 are shown in FIG. 1 as lace appearing designs, the strips 12 can alternatively include designs more suitable for a man. It is further appreciated that the strips 12 could alternatively include designs suitable for a child of either gender.

In some embodiments, the undergarment body 11 includes an elastic rim surrounding each leg hole 16.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. A decorative disposable undergarment, comprising:
a undergarment body defining a disposable lower body undergarment that includes at least one integrated pad, wherein said undergarment body includes a top edge and an exterior surface;
a plurality of discrete elastic retaining strips attached to the exterior surface, wherein each of said retaining strips encircle the undergarment body and is positioned at a distinct, non overlapping vertical position on the undergarment body; and
a decorative hanging portion attached to the lowermost retaining strip, wherein said hanging portion extends downwardly below at least a portion of the pad.

2. The decorative disposable undergarment of claim 1, wherein said retaining strips are sewn to the undergarment body.

3. The decorative disposable undergarment of claim 2, wherein the uppermost retaining strip is adjacent to the top edge.

4. The decorative disposable undergarment of claim 2, wherein at least two of the retaining strips are contiguous.

5. The decorative disposable undergarment of claim 1, wherein the uppermost retaining strip is adjacent to the top edge.

6. The decorative disposable undergarment of claim 5, wherein at least two of the retaining strips are contiguous

7. The decorative disposable undergarment of claim 1, wherein at least two of the retaining strips are contiguous.

8. A decorative disposable undergarment, comprising:
a undergarment body defining a disposable lower body undergarment that includes at least one integrated pad, wherein said undergarment body includes a top edge and an exterior surface;
a plurality of discrete elastic retaining strips attached to the exterior surface, wherein each of said retaining strips encircle the undergarment body and is positioned at a distinct, non overlapping vertical position on the undergarment body;
a decorative hanging portion attached to the lowermost retaining strip, wherein said hanging portion extends downwardly below at least a portion of the pad; and
wherein said retaining strips are sewn to the undergarment body, the uppermost retaining strip is adjacent to the top edge, and at least two of the retaining strips are contiguous.
